(19) United States
(12) Patent Application Publication
(10) Pub. No.: US 2021/0020305 A1
(43) Pub. Date: Jan. 21, 2021

(54) SYSTEM AND METHOD FOR SCHEDULING AND MANAGING SERVICES

(71) Applicant: Gregory W. Ray, Los Angeles, CA (US)

(72) Inventor: Gregory W. Ray, Los Angeles, CA (US)

(21) Appl. No.: 16/927,322

(22) Filed: Jul. 13, 2020

Related U.S. Application Data
(60) Provisional application No. 62/846,860, filed on May 13, 2019.

(51) Int. Cl. G16H 40/20; G16H 10/60; G06N 20/00; G06K 9/62

(52) U.S. Cl. CPC G16H 40/20; G06K 9/6256; G06N 20/00; G16H 10/60

(57) ABSTRACT

The present disclosure describes a system for data and signal routing for scheduling and managing services. The system has at least one client device associated with a user, wherein the at least one client device that allows a user to input a request to a first service provider, wherein the first service provider is a healthcare clinic; a network for receiving the input request to the first service provider; an integrated service management server in communication with the network, wherein the integrated management server receives the input request from the first service provider, and outputs a request to a second service provider, wherein the second service provider is a transport company; a registration module residing on the integrated management server that enables the user to register with the integrated management server by inputting personal credentials, wherein the registration module outputs the personal credentials to a patient database via the network; a scheduling module residing on the integrated management server, wherein the scheduling module arranges, based on the input request and the personal credentials on the database, a clinic appointment for the user and a transportation type to the clinic for the user; wherein the transportation type is one of a plurality of different types of vehicles in a vehicles fleet.

### CROSS-REFERENCE TO RELATED APPLICATIONS

This application claims the benefit of U.S. Provisional Patent Application Ser. No. 62/846,860 filed May 13, 2019 entitled System and Method System and Method for Scheduling and Managing Services, the entire contents of which are incorporated by reference herein for all purposes.

### FIELD OF THE INVENTION

The present invention generally relates to a system and method for data a signal routing for scheduling and managing services. More specifically, the present invention relates to a system and method that integrates a transport service and a business in the healthcare service industry and allows scheduling and management of appointments and transport tasks within the business (e.g., healthcare provider) utilizing the transport service.

### BACKGROUND

In general, it is necessary to get appointment before meeting any service provider such as entertainment service, counseling service, educational service and healthcare providers such as hospitals and doctors. Healthcare service providers is one area where a patient or user needs to book an appointment with a doctor who may then subsequently require the patient to book an appointment with a specialist, hospital, blood lab, X-ray clinic or other scanning facility. Appointment booking via telephone or in person within a specific group of healthcare practitioners results in treatment with the available physician, fee schedules and treatment procedures. Booking appointments in the healthcare industry requires receptionist working time and the number of calls being handled by the receptionist in the clinic which is inefficient.

A massive problem for healthcare providers is the cost of patients missing the aforementioned appointment. The total cost of missed healthcare appointments in the United States every year is an astronomical $150 billion (estimated in 2017). Each open, unused time slot costs a physician 60 minutes and $200 on average. Factoring in that no-show rates are as high as 30% nationwide, this $150 billion figure is will continue to grow. Even with the many high-tech digital reminders at our disposal, this problem still exists. Furthermore, a missed medical appointment could pose serious health risks for patients as it could mean the difference between catching a disease early on or too late. In addition, an inefficient scheduling process can wreak havoc and raise stress levels for both a health systems' staff and patients.

This has forced some healthcare providers to force patients go blind queue up, and wait in line up to several hours, while the actual treatment time is only a few minutes for some minor ailments such as fever. This is incredibly inconvenient to patients and leads to patients not seeking treatment at all.

By their very nature, patients have ailments or are at the latter stages of life which may make it exceedingly difficult for the patient to get to their appointment. While attempts have made to restructure the process of scheduling appointments for patients in healthcare industry, Patients may, in some systems. connect with the organization via web and schedule their appointment by themselves. However, this type of scheduling appointment by patients have drawbacks such as security issue while sharing the medical information over internet, however, these patients still miss their appointments at the same high rate.

Further, many times a sick patient will arrive at a clinic that is not suited for their specific illness, and they must be transported to a different facility. As an example, a patient with appendicitis may go to an urgent care that does not have an ultrasound or Mill to diagnoses such an illness. In this case valuable time is waste sending the patient to a new facility.

Therefore, there is a need for a system and method that provides an integrated service combining a transport service and a business such as a healthcare service, to enable a user to schedule and manage appointment with the business utilizing the transport service.

### SUMMARY OF THE INVENTION

The following summary of the invention is provided in order to provide a basic understanding of some aspects and features of the invention. This summary is not an extensive overview of the invention and as such it is not intended to particularly identify key or critical elements of the invention or to delineate the scope of the invention. Its sole purpose is to present some concepts of the invention in a simplified form as a prelude to the more detailed description that is presented below.

To achieve the foregoing and other aspects and in accordance with the purpose of the invention, a system and method that integrates a transport service and a business in the healthcare service industry and allows scheduling and management of appointments and transport tasks within the business (e.g., healthcare provider) utilizing the transport service.

Accordingly, this platform provides a system and method that provides integrated services combining a transport service and a business such as a healthcare service.

Further, the platforms provide a HIPPA compliant networked system that is in communication with a fleet of autonomous self-driving automobiles that picks up patients for a healthcare appointment.

Further, the platform provides a HIPPA compliant networked system in which the fleet is given relevant information about patient to ensure the correct automobile is used for the patent needs (e.g., wheelchairs).

Further, the platform provides a system and method that enables a user to schedule and manage appointment with the business utilizing the transport service.

Further, the platform provides a system and method that directly links the user and the healthcare provider from pickup to drop off point to ensure that the patient arrives on time and availability of a person concerning the business.

The system comprises one or more processors, dedicated servers, a memory, and specialized date routing and communications protocol via the dedicated servers in communication with processors, user smart devices and third-party providers. The memory stores program instructions executable by the processors to implement an integrated service. The system enables a user to schedule an appointment with a first service and enables the user to schedule an appointment with a second service for transport. The system enables the first service provider to track a status of the appointment of the user, wherein the first service is a healthcare service and wherein the second service is a transportation service.

In one embodiment, the first service is provided by a first service provider and a second service is provided by a second service provider. In one embodiment, the system comprises on both the user side and third-party side, estimated time of arrival. In one embodiment, the transportation service is a shared transportation service and may be an autonomous vehicle for transport. In one embodiment, the first service at least one of medical service, entertainment service, counseling service, and educational service.

In one embodiment, a system for data and signal routing for scheduling and managing services is provided. The system comprises at least one client device associated with a user, wherein the at least one client device that allows a user to input a request to a first service provider, wherein the first service provider is a healthcare clinic; a network for receiving the input request to the first service provider; an integrated service management server in communication with the network, wherein the integrated management server receives the input request from the first service provider, and outputs a request to a second service provider, wherein the second service provider is a transport company; a registration module residing on the integrated management server that enables the user to register with the integrated management server by inputting personal credentials, wherein the registration module outputs the personal credentials to a patient database via the network; a scheduling module residing on the integrated management server, wherein the scheduling module arranges, based on the input request and the personal credentials on the database, a clinic appointment for the user and a transportation type to the clinic for the user; wherein the transportation type is one of a plurality of different types of vehicles in a vehicles fleet.

In another embodiment, a non-transitory computer-readable medium for storing instructions that, when executed on one or more processors, cause the one or more processors to: generate, for display on a client device graphical user interface (GUI) associated with at least one user, an input screen that allows a user to input a request to a first service provider, wherein the first service provider is a healthcare clinic be completed by the user; receive the input from the client device GUI at an integrated server via a network, and in response, output a request to a second service provider, wherein the second service provider is a transport company; receive user personal credentials associated with the user; output the patient credentials to a patient database via the network; arrange, based on the input request and the personal credentials on the database, a clinic appointment for the user and a transportation type to the clinic for the user; wherein the transportation type comprises one of a plurality of different types of vehicles in a vehicles fleet, wherein the plurality of different types of vehicles in fleet comprise a car, a handicap accessible van, and an ambulance, wherein the vehicles are autonomous vehicles.

A method for data and signal routing for scheduling and managing services incorporated in a system including a client device, and a server in communication with the client device, wherein the server comprises a memory to store instructions and a processor coupled with the memory to process the stored instructions, the method comprising the steps of generating, for display on a client device graphical user interface (GUI) associated with at least one user, an input screen that allows a user to input a request to a first service provider, wherein the first service provider is a healthcare clinic be completed by the user; receiving the input from the client device GUI at an integrated server via a network, and in response, output a request to a second service provider, wherein the second service provider is a transport company; receiving user personal credentials associated with the user; outputting the patient credentials to a patient database via the network; arranging, based on the input request and the personal credentials on the database, a clinic appointment for the user and a transportation type to the clinic for the user wherein the transportation type comprises one of a plurality of different types of vehicles in a vehicles fleet.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 illustrates a platform schematic diagram of a system for scheduling and managing services, according to an embodiment of the present invention.

FIG. 2 illustrates an integrated service management server, according to an embodiment of the present invention.

FIG. 3 shows an exemplary screenshot of a registration user interface, according to an embodiment of the present invention.

FIG. 4 shows an exemplary screenshot of a user interface for displaying list of healthcare providers, according to an embodiment of the present invention.

FIG. 5 shows an exemplary screenshot of a user interface for displaying map data and vehicle data, according to an embodiment of the present invention.

FIG. 6 shows an exemplary screenshot of a user interface for displaying various features provided by an integrated service of the system, according to an embodiment of the present invention.

FIG. 7 shows an exemplary screenshot of a user interface for displaying upcoming scheduled appointments, according to an embodiment of the present invention.

FIG. 8 shows an exemplary screenshot of a user interface for displaying past scheduled appointments, according to an embodiment of the present invention.

FIG. 9 shows an exemplary screenshot of a user interface for displaying upcoming scheduled appointment details, according to an embodiment of the present invention.

FIG. 10 shows an exemplary screenshot of a user interface for displaying trip details of scheduled appointments, according to an embodiment of the present invention.

FIG. 11 shows an exemplary screenshot of a user interface for displaying vehicle types, according to an embodiment of the present invention.

FIG. 12 shows an exemplary screenshot of user interface for displaying trip history, according to an embodiment of the present invention.

FIG. 13 shows an exemplary screenshot of user interface for displaying trip details to a transport service provider, according to an embodiment of the present invention.

FIG. 14 shows an exemplary screenshot of user interface displaying trip history, according to another embodiment of the present invention.

FIG. 15 illustrates a flowchart of a method for scheduling and managing services, according to an embodiment of the present invention.

FIG. 16 illustrates a system diagram for scheduling and managing services, according to an embodiment of the present invention.

Other features, advantages, and aspects of the present invention will become more apparent and be more readily understood from the following detailed description, which should be read in conjunction with the accompanying drawings.

### DETAILED DESCRIPTION OF THE INVENTION

The present invention is best understood by reference to the detailed figures and description set forth herein.

It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

Reference will now be made in detail to various embodiments. Each example is provided by way of explanation and is not meant as a limitation and does not constitute a definition of all possible embodiments. The described embodiments are to be considered in all respects only as illustrative and not restrictive. For purposes of illustrating features of the embodiments, a simple example will now be introduced and referenced throughout the disclosure. Those skilled in the art will recognize that this example is illustrative and not limiting and is provided purely for explanatory purposes. An example of a computing system environment is disclosed. The computing system environment is not intended to suggest any limitation as to the scope of use or functionality of the system and method described herein. Neither should the computing environment be interpreted as having any dependency or requirement relating to any one or combination of components illustrated in the exemplary operating environment.

Embodiments of the disclosure are operational with numerous other general purposes or special purpose server and computing system environments or configurations. Examples of well-known computing systems, environments, and/or configurations that may be suitable for use with the systems and methods described herein include, but are not limited to, smartphones, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The embodiments of the disclosure may be described in the general context of computer-executable instructions, such as program modules, being executed by a computer. Generally, program modules include routines, programs, objects, components, data structures, etc. that perform particular tasks or implement particular abstract data types. The systems and methods described herein may also be practiced in specialized distributed computing environments where tasks are performed by remote processing devices that are linked through a communications network. In a specialized distributed computing environment, program modules may be located in both local and remote computer storage media including memory unit or storage devices. Tasks performed by the programs and modules are described below and with the aid of figures. Those skilled in the art can implement the exemplary embodiments as processor executable instructions, which can be written on any form of a computer readable media in a corresponding computing environment according to this disclosure.

Components of computer may include, but are not limited to, a processing unit, a system memory, and a system bus that couple various system components including the system memory to the processing unit. The system bus may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

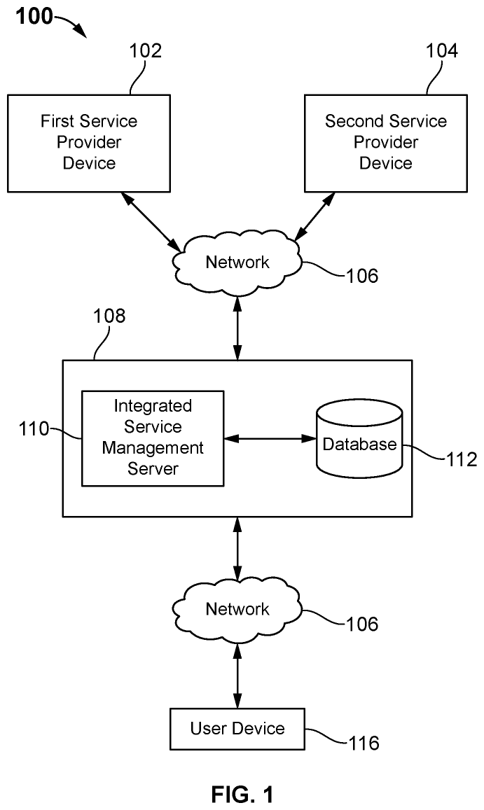

Referring now to FIG. 1, the system or platform comprises an integrated service management system 100, a first service provider device 102 associated with a first service provider, a second service provider device 104 associated with a second service provider, and a user device 116 associated with a user. The integrated service management system 108 comprises an integrated service management server 110 and a database 112. The first service provider device 102, the second service provider device 104 and the user device 116 are in communication with the integrated service management server 110 via a network 106 which is HIPPA compliant in some embodiments.

In an embodiment, the user device 116 is a mobile phone running a mobile application but may be desktop, a laptop, a tablet, a mobile phone, and mobile and/or handheld electronic devices. In an embodiment, the second service provider device 104 is a mobile phone running a mobile application but may be at least one of a desktop, a laptop, a tablet, a mobile phone, and mobile and/or handheld electronic devices. In one embodiment, the network 106 could be Wi-Fi network, WiMax network, and wireless local area network.

In one embodiment, the database 112 may be accessible by the integrated service management server 110. The database 112 may be integrated into the integrated service management server 110 or separate from it. In some embodiments, the database 112 resides in the connected server 110 or in a cloud computing service that is, in some embodiments, HIPPA compliant. Regardless of location, the database 112 comprise a memory to store and organize certain data for use by the integrated service management server 110. In some embodiments, the database stores plurality of user related information, one or more service providers related information and an integrated service management algorithm, and machine learning techniques to improve performance over time.

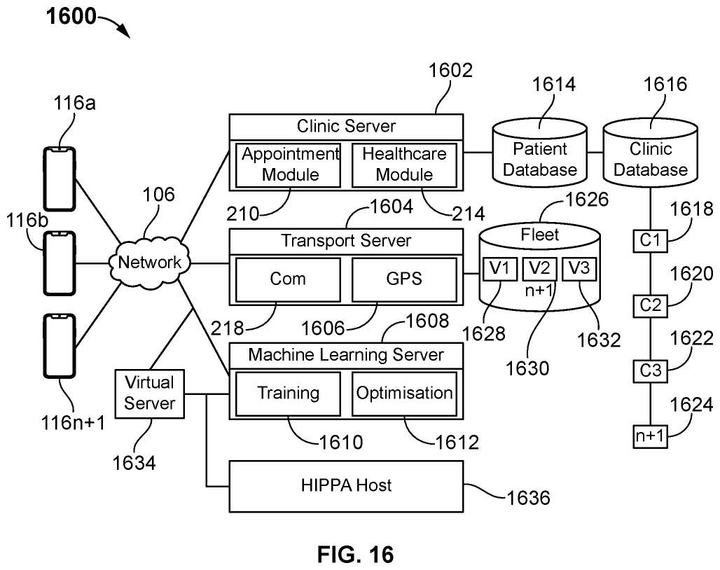

In one embodiment, the integrated service management server 110 comprises special purposes server and data routing schemes as shown in FIG. 16. The integrated service management server 110 may operate via a mainframe, a supercomputer, a server farm, and so forth. In one embodiment, the plurality of computers is in communication with each other, via networks. The communication can be via a software application, a mobile app, a browser, an OS, and/or any combination thereof.

In one embodiment, the first service provider provides a first service and a second service provider provides a second service. In one embodiment, the first service is at least one of medical service, entertainment service, counseling service, and educational service and the second service is a transportation service. In one embodiment, the transportation service is a shared transportation service. In one embodiment, the transportation service comprises an autonomous vehicle for transport. The server 110 enables the user to schedule an appointment with the first service and further to enable the user to schedule an appointment with the second service for transport. The server 110 further enables the first service to track a status of the appointment of the user, change appointments unilaterally via notification and the lie.

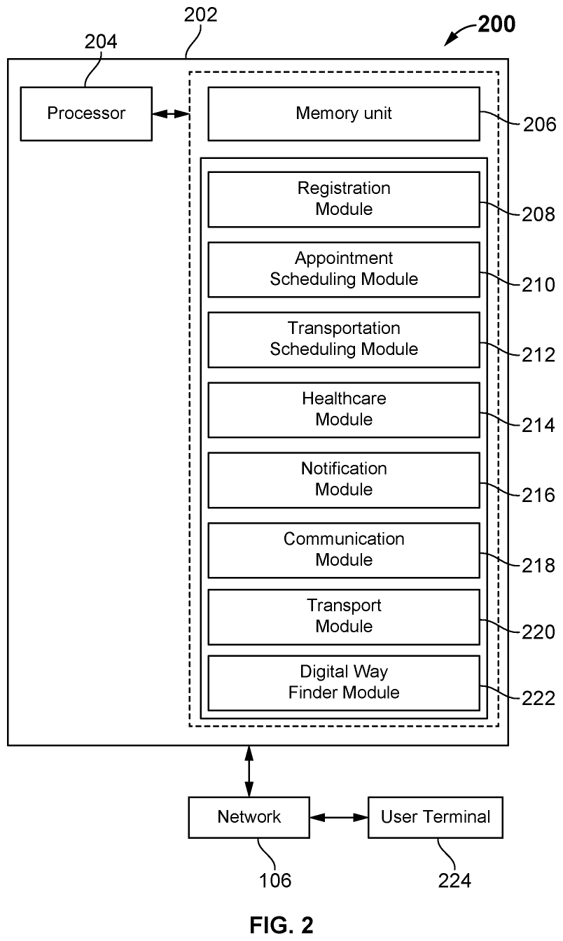

In another embodiment, the first service is a transport service and a second service is a healthcare service. In one embodiment, the user could be a patient or a care provider of the patient. The first service provider device 102 is associated with a physician or an administrator aiding physician and the second service provider device 104 is associated with a driver or autonomous vehicle responsible for transport of the patient or the care provider. The integrated service management server 110 enables the patient to schedule an appointment with the first service and schedule an appointment with the second service for transport. The integrated service management server 110 further enables the first service to track a status of the appointment of the patient, which includes estimated time of arrival of patient. In one embodiment, the status includes estimated time of arrival (ETA). In embodiments, the communications protocols identify the needs of the patients so that the drive or vehicle has the necessary services available for the patient. A block diagram 200 of the integrated service management server 110 is detailed and illustrated in FIG. 2.

Referring to FIG. 2, the server 110 comprises a processor 204 and a memory unit 206. The memory unit 206 comprises a set of program modules. The set of program modules, which operate via the specialized servers shown in FIG. 16 comprises a registration module 208, an appointment scheduling module 210, a transportation scheduling module 212, a healthcare module 214, a notification module 216, a communication module 218, a transport module 220 and a digital way finder module 222. In one embodiment, the user, the healthcare provider, and the transport service provider access server utilizing a user terminal 224 via a network 106.

The registration module 208, executed by the processor 204, enables a user to register with the server 110 by inputting credentials including, but not limited to, first name, last name, email address, phone number, medical record number, healthcare organizations, appointment date, appointment time, password, and touch ID log-in. A healthcare provider may enable registration for its users/patients automatically to avoid patients having to input the information themselves, as some patients are not able to do so or use mobile applications. The registration module allows the user to provide communication protocol with the user's health insurances provider (HMO or PPO) in a HIPPA compliant manner and provides patient background allergies, and other requirements.

The appointment scheduling module 210, executed by the processor 204, enables the user to view a list of healthcare service provider retrieved from the database. In one embodiment, the user is provided with an option to add new service providers to their existing list of service providers. The appointment scheduling module further enables the user to book an appointment with the features including, but not limited to, calendar for date navigation, appointment addition option, list of unavailable appointments indicated by a color, and appointment cancellation and rescheduling options.

The transportation scheduling module 212, executed by the processor 204, is enables the user or the healthcare provider to book a vehicle for transport or choose a transport by inputting details, including, but not limited to, vehicle type, departure time, departure location and destination location. The healthcare module 214, executed by the processor 204, provides or manages details of service providers, including, but not limited to, choice of working hours, lunch break hour's selection, off-days selection (Saturday/Sunday/Holidays), history keeping (30-60-90) days, messages such as special announcements to be displayed to the clients, etc. Each may be in communication with an autonomous vehicle.

The notification module 216, executed by the processor 204, sends notification in case of change in appointments, cancellation of appointments, revised date and time from any end of user such as a patient, or a service provider. In one embodiment, the notification could be sent by means, including, but not limited to, SMS, email, or push message. The communication module 218, executed by the processor 204, enables communication between the transport service provider, healthcare service provider and the user. The transport module 220, executed by the processor 204, provides trip details to the transport service provider. The digital way finder module 222, executed by the processor 204, provides map data to the transport service provider for transport of the user.

The healthcare module 214 allows the user to input current conditions, and may comprise a telehealth sub-module component for communication with a physician or clinician.

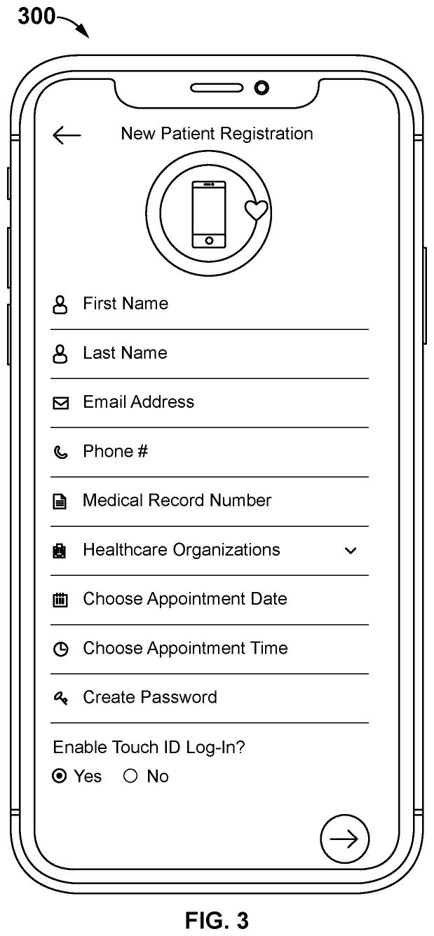

Referring now FIG. 3 illustrates a screenshot 300 showing, by way of an example, a user interface (UI) for registering a user on the database through the registration module 208 in accordance with an embodiment of the present system. The registration module 208 allows the user to register on the database by inputting the credentials. Examples of credentials comprises first name, last name, email address, phone number, medical record number, healthcare organizations, appointment date, appointment time, password, and touch ID log-in.

The UI allows the user to enter the email address, the password, and a first name respectively for accessing in the database through register option. In another embodiment of the present system, the user may further access in the database by using social media details such as Facebook credentials through social media option.

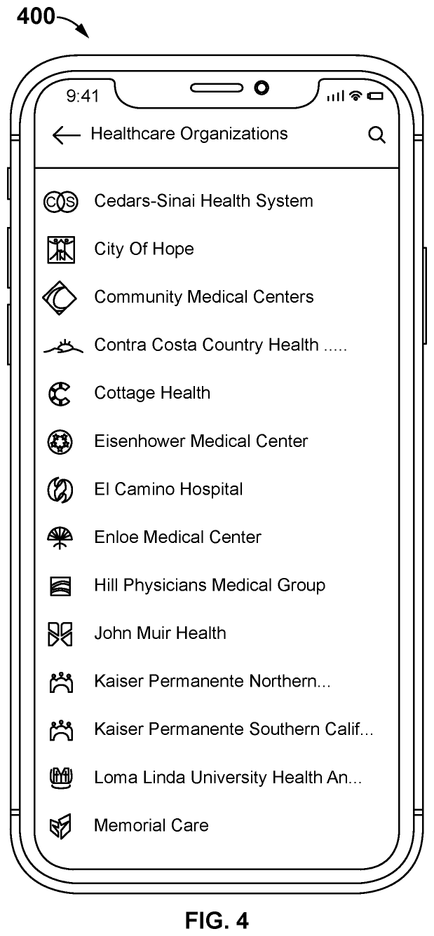

FIG. 4 illustrates a screenshot 400 showing, by way of an example, an UI for displaying list of healthcare providers in accordance with an embodiment of the present system. The appointment scheduling module 210 allows the user to select a preferred healthcare provider from the displayed list.

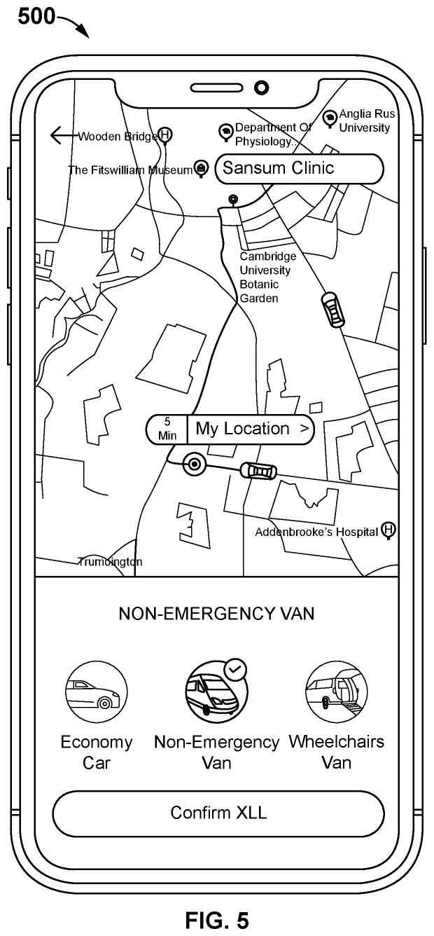

FIG. 5 illustrates a screenshot 500 showing, by way of an example, an UI for displaying map data and vehicle data in accordance with an embodiment of the present system. The transport scheduling module 212 allows the user to select a mode of transport or a vehicle type. The transport module 220 is further configured to enable the user to select pickup location and destination location, for example textually or graphically on a map.

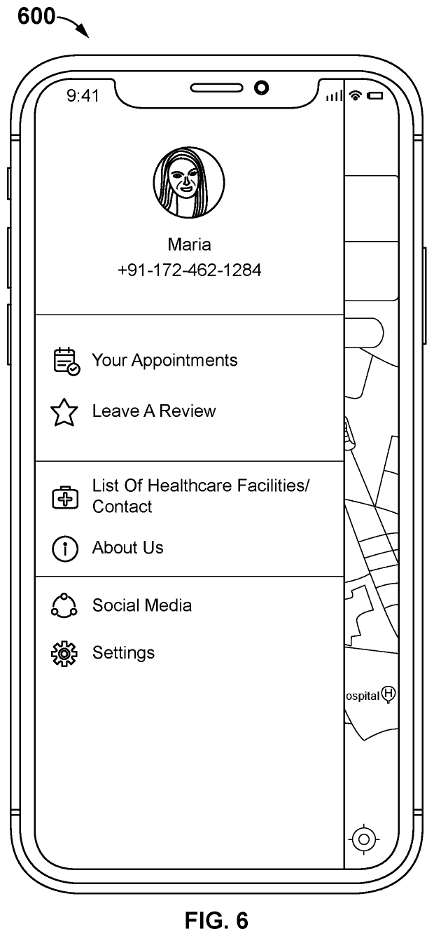

FIG. 6 illustrates a screenshot 600 showing, by way of an example, an UI for displaying various features provided by an integrated service in accordance with an embodiment of the present system. In an embodiment, the features include, but not limited to, scheduled appointments, review, list of healthcare facilities/contact, social media and settings.

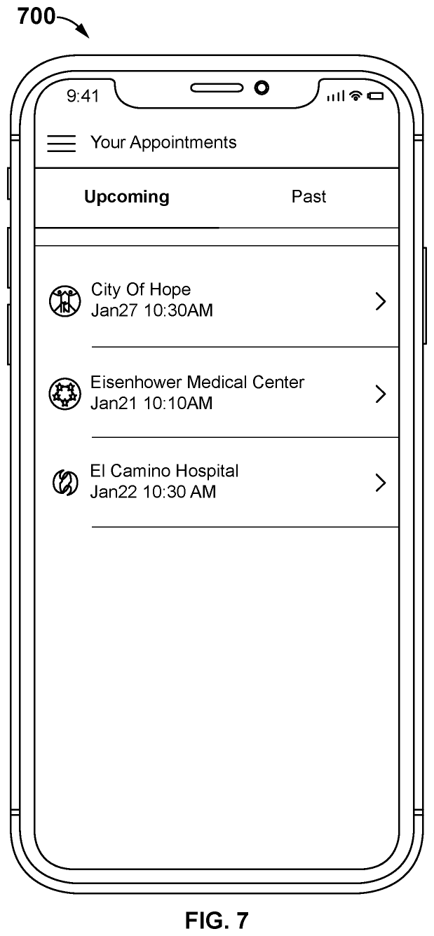

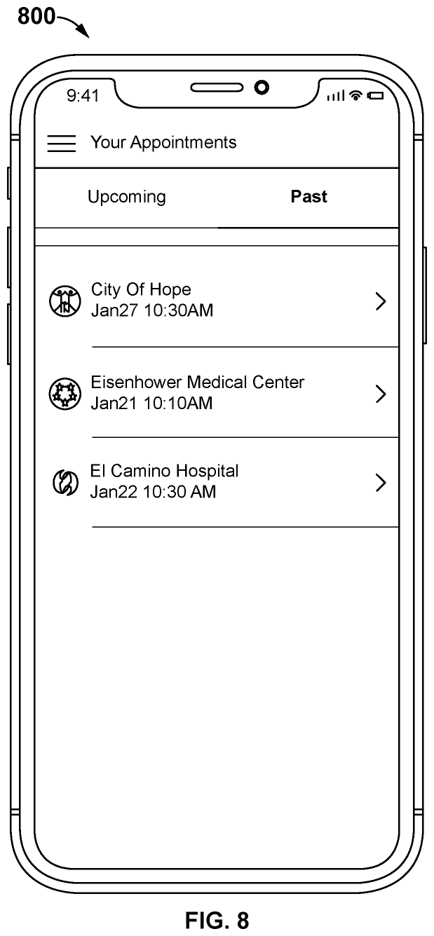

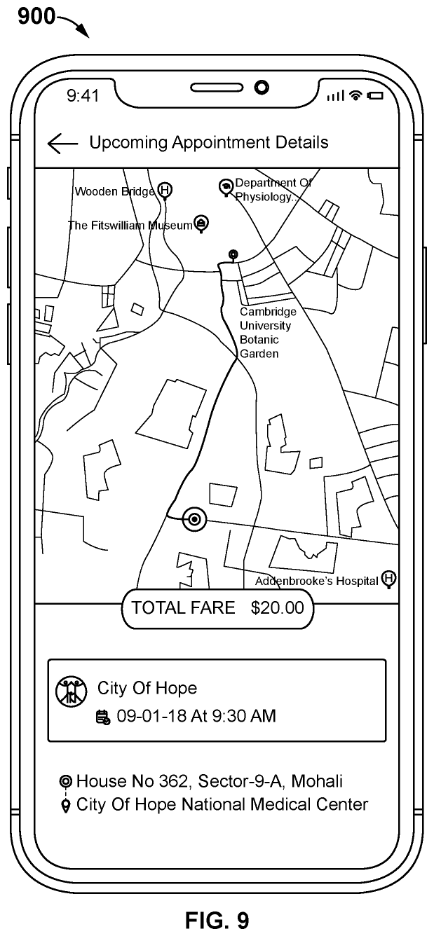

FIG. 7 illustrates a screenshot 700 showing, by way of an example, an UI for displaying upcoming scheduled appointments in accordance with an embodiment of the present system. FIG. 8 illustrates a screenshot 800 showing, by way of an example, an UI for displaying past scheduled appointments in accordance with an embodiment of the present system. FIG. 9 illustrates a screenshot 900 showing, by way of an example, an UI for displaying upcoming scheduled appointment details in accordance with an embodiment of the present system. On selecting at least one scheduled appointment, the UI is configured to display details of the respective selected appointment, which includes but not limited to, location of the healthcare provider on the map, and total fare for transport.

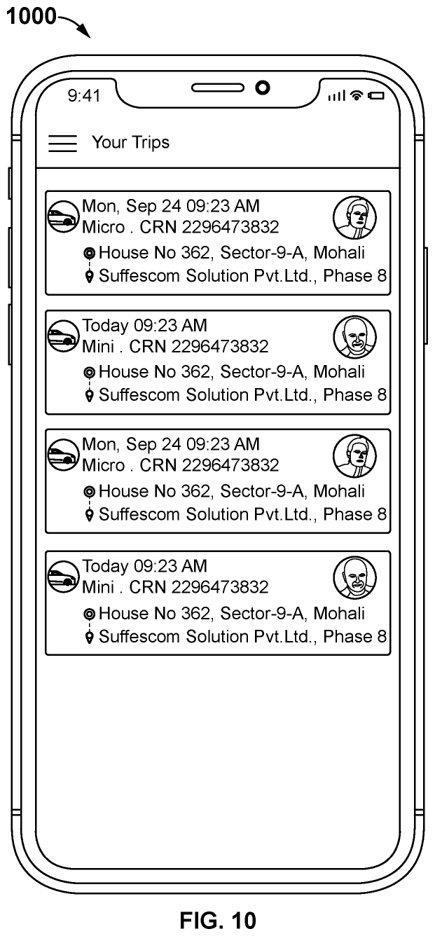

FIG. 10 illustrates a screenshot 1000 showing, by way of an example, an UI for displaying trip details in accordance with an embodiment of the present system. The trip details include, but not limited to, date of the appointment, vehicle type, pick up location, and destination location.

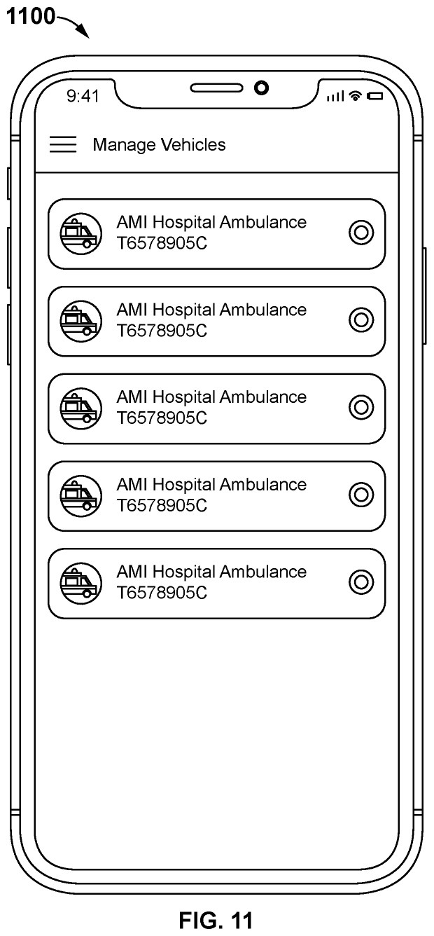

FIG. 11 illustrates a screenshot 1100 showing, by way of an example, an UI for displaying vehicle types in accordance with an embodiment of the present system.

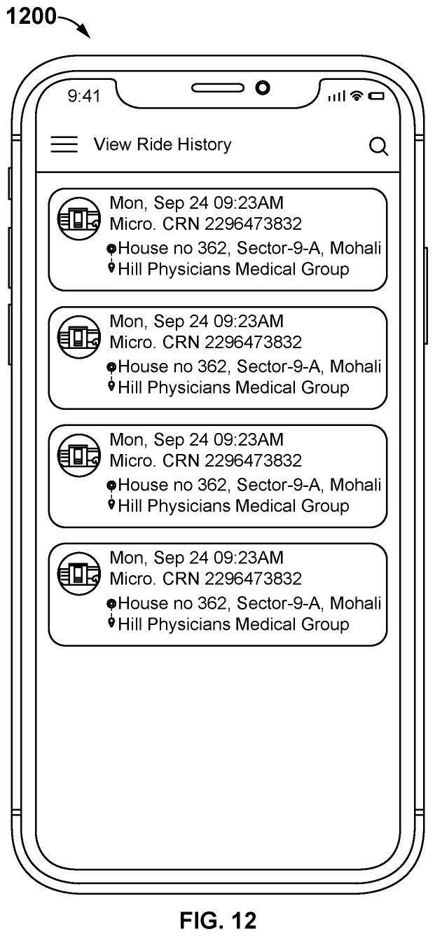

FIG. 12 illustrates a screenshot 1200 showing, by way of an example, an UI for displaying trip or ride history in accordance with an embodiment of the present system.

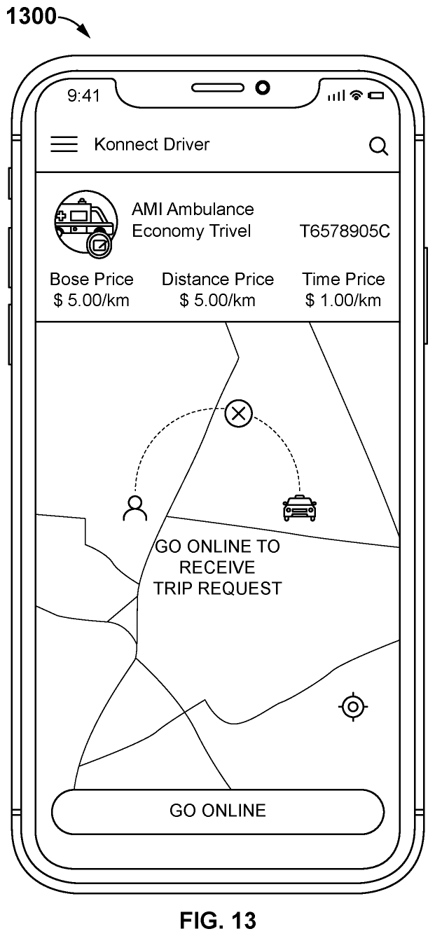

FIG. 13 illustrates a screenshot 1300 showing, by way of an example, an UI for displaying trip details to a transportation service provider in accordance with another embodiment of the present system.

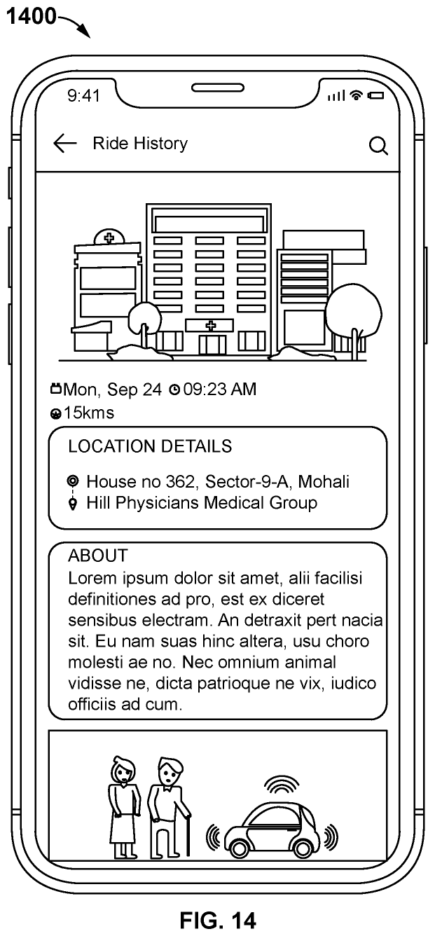

FIG. 14 illustrates a screenshot 1400 showing, by way of an example, an UI for displaying trip history in accordance with another embodiment of the present system.

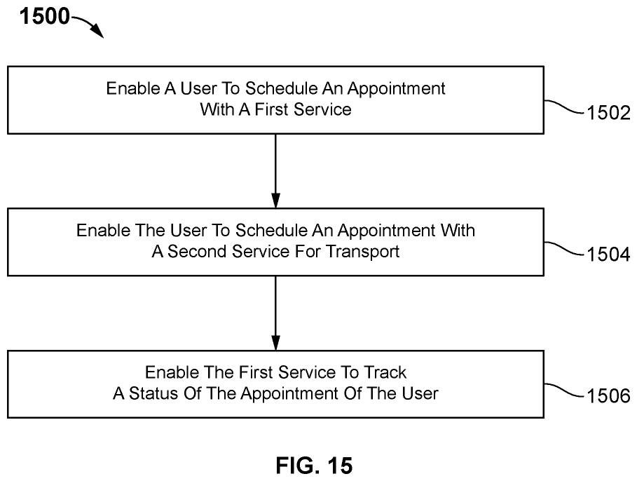

FIG. 15 exemplarily illustrates a flowchart of a method 1500 for scheduling and managing services, according to an embodiment of the present system. In one embodiment, the method 1500 is implemented in a system comprising one or more processors, a memory coupled to the one or more processors, wherein the memory stores program instructions executable by the one or more processors to implement an integrated service. At step 1502, the system is configured to enable a user to schedule an appointment with a first service. At step 1504, the system is configured to enable the user to schedule an appointment with a second service for transport. At step 1506, the system is configured to enable the first service to track a status of the appointment of the user, wherein the first service is a healthcare service and wherein the second service is a transportation service.

Referring now to FIG. 16, a specialized server schematic diagram is shown generally at 1600. User devices 116a, 116b, and 116n+1 are connected to multiple servers via network 106. A clinic server 1602, a transport server 1604, and a machine learning server 1608 are communicatively coupled to the user devices in a bidirectional manner via network 108. The clinic server 1602 comprises appointment scheduler 210 and healthcare module 214. The clinic server 1602 is in communication with patient database 614, which stores patient historical data from the user's health insurances provider (HMO or PPO) or doctor/clinic in a HIPPA compliant manner and also current patient data from the user's inputs into the platform (e.g., user inputs symptoms on the GUI), and provides patient background and any current symptoms, allergies, and other needs. A clinic database 1616 is in communication with the patient database and clinic server 1602. In operation, based on the patients background and current needs, the clinic server 1602 parses the input data to ensure the patient is transported via the transportation scheduling module to one of a plurality of clinics C1, C2, C-3 or C (n+1) that can provide the healthcare requirements whether it be C1, C2, C-3 or C (n+1) shown at reference numerals 1618, 1620, 1622, and 1624. As an example, C1 may not have an MRI, whereas C2 has an MRI. C3 may have anti-venom whereas C1 and C2 don't, and so on. Critically, the clinic server 1602 and transport service 1604 are in communication with each other via the network to ensure proper location management for the patient at the end of user device 116. As an example, if a patient has a sharp pain in the lower right quadrant of their abdomen, the system will ensure the patient goes to a clinic or hospital that has the appropriate medical equipment to treat appendicitis. The clinic database 1616 comprises a list of equipment surgeries that the hospital or clinic can perform. The healthcare module 214 works with the patient database 1614 and the inputs to ensure the patient is matched with an appropriate clinic based on the combination of variables and factors.

Approximately concurrently, the transport server 1604 is provided with the clinic decision via communications module 218 and will rely on GPS module 1606 to pick up the patients and bring them to the appropriate clinic. The transport server module 1604 is in communication with a fleet of vehicles 1626 which may be driven by a person or may be autonomous. The fleet may comprise V1, V2, V3 n+1 denoted by reference numerals 1628, 1630, and 1632. In operation, based on the patient's background and current needs, the transport server parses the input data to ensure the patient is transported in a vehicle that can provide the healthcare requirements whether it be V1, V2, V3 (n+1). As an example, V1 may not wheelchair ramp access, whereas V2 has wheelchair ramp access. V3 may be an ambulance in case of an emergency, and so on. Critically, the transport server 1604 and clinic server 1604 are in communication with each other via the network to ensure proper vehicle and location management for the patient at the end of user device 116. The fleet 1626 may be in a database of vehicles at the station, and work with the patient database and the inputs to ensure the patient is matched with an appropriate vehicle based on the combination of patient variables and factors. In some embodiments, the system may utilize photograph recognition to easily input types of vehicles and types of equipment into the databases. In this way, the system recognizes which vehicle and instrument and automatically populates the database.

Due to the large amount of data, data from the platform is parsed and sorted at virtual server 1634, then sent to the machine learning module 1608. A loop is created between the machine learning module 1608 and virtual server 1634 to continuously train nodes on training module 1610 on a neural network (machine learning), or on other embodiments, execute a random forest algorithm to run the predictive analyses on the data to optimize vehicle and clinical end points depending upon patient needs for optimization module 1612.

HIIPA host 1636 is a compliant server that follows the guidelines defined by HIPAA to prevent medical record information data breaches and thus comprises physical data storage security, physical data destruction security, data access security, data integrity security, data transfer security and data breach reporting

Advantageously, the system integrates appointment booking for healthcare and transport service in a single platform, that allows a patient to book their ride and provide ETA of ride share vehicle. The system enables the service provider to view and track patient's location and ETA via GPS from vehicle. The present system directly links the patient and the healthcare provider from pickup to drop off and ensures a patient arrives on time. The present system also Solves and reduces number of missed appointments, removes the transportation barriers for patients and allows transparency on real time information such as estimated time of arrival, patient pick up confirmation, etc.

The present system will make it easier for patients to get to appointments and make them less likely to miss an appointment whilst providing them appropriate transport and care.

Preferred embodiments of this invention are described herein, including the best mode known to the inventors for carrying out the invention. It should be understood that the illustrated embodiments are exemplary only and should not be taken as limiting the scope of the invention.

Although specific features of various embodiments of the invention may be shown in some drawings and not in others, this is for convenience only. In accordance with the principles of the invention, the feature(s) of one drawing may be combined with any or all of the features in any of the other drawings. The words “including,” “comprising,” “having,” and “with” as used herein are to be interpreted broadly and comprehensively and are not limited to any physical interconnection. Moreover, any embodiments disclosed herein are not to be interpreted as the only possible embodiments. Rather, modifications and other embodiments are intended to be included within the scope of the appended claims.

I claim:

1. A system for data and signal routing for scheduling and managing services, the system comprising:
at least one client device associated with a user, wherein the at least one client device comprises a graphical user interface (GUI) that allows a user to input a request to a first service provider, wherein the first service provider is a healthcare clinic;
a network for receiving the input request to the first service provider;
an integrated service management server in communication with the network, wherein the integrated management server receives the input request from the first service provider, and outputs a request to a second service provider, wherein the second service provider is a transport company;
a registration module residing on the integrated management server that enables the user to register with the integrated management server by inputting personal credentials, wherein the registration module outputs the personal credentials to a patient database via the network;
a scheduling module residing on the integrated management server, wherein the scheduling module arranges, based on the input request and the personal credentials on the database, a clinic appointment for the user and a transportation type to the clinic for the user;
wherein the transportation type comprises one of a plurality of different types of vehicles in a vehicles fleet.

2. The system of claim 1, wherein the plurality of different types of vehicles in fleet comprise a car, a handicap accessible van, and an ambulance, and wherein the vehicles are autonomous vehicles.

3. The system of claim 1, further comprising a healthcare module to enable the user to input a current condition, and a telehealth sub-module for communication with a physician or clinician, wherein the scheduling module utilizes current conditions to select the transportation type to the clinic for the user.

4. The system of claim 1, further comprising a transportation scheduling module residing on the server to enable the user or the clinician to book one of the types of vehicles in the fleet.

5. The system of claim 4, further comprising a clinic database, wherein the clinic database stores medical equipment types, and wherein the transportation scheduling module is in communication with the clinic database, and based on the personal credentials and current conditions, schedule the patient at one of a plurality of clinics.

6. The system of claim 1, further comprising a notification module residing on the integrated management server, wherein the notification module sends notification to the user and the clinic of a change of an appointment, a cancellation of the appointments, and via GPS, arrival of the user.

7. The system of claim 1, further comprising a digital way finder module in communication with the transport module and user device to provide map data to the transport service for transport of the user.

8. The system of claim 1, further comprising:
a clinic server in communication with the patient database and clinic database and the user device;
a transport server in communication with the clinic server and the user device;
a virtual server in communication with the clinic database and patient database, wherein the virtual server parses and sorts data to transform the data for output;
a machine learning server to receive the data from the virtual server, wherein the machine learning module comprises:
a training module for collecting the data and update the machine learning module thereby providing a loop; and
an optimizer module to analyze the data and reconfigure the transport server and clinic server to improve system performance.

9. A non-transitory computer-readable medium for storing instructions that, when executed on one or more processors, cause the one or more processors to:
generate, for display on a client device graphical user interface (GUI) associated with at least one user, an input screen that allows a user to input a request to a first service provider, wherein the first service provider is a healthcare clinic be completed by the user;
receive the input from the client device GUI at an integrated server via a network, and in response, output a request to a second service provider, wherein the second service provider is a transport company;
receive user personal credentials associated with the user;
output the patient credentials to a patient database via the network;
arrange, based on the input request and the personal credentials on the database, a clinic appointment for the user and a transportation type to the clinic for the user;
wherein the transportation type comprises one of a plurality of different types of vehicles in a vehicles fleet.

10. The non-transitory computer-readable medium of claim 9, wherein the plurality of different types of vehicles in fleet comprise a car, a handicap accessible van, and an ambulance, wherein the vehicles are autonomous vehicles.

11. The non-transitory computer-readable medium of claim 9 for storing instructions that, when executed on one or more processors, cause the one or more processors to further:
enable the user to input a current conditions and a telehealth sub-module for communication with a physician or clinician, wherein the scheduling module utilizes current conditions to select the transportation type to the clinic for the user

12. The non-transitory computer-readable medium of claim 9 for storing instructions that, when executed on one or more processors, cause the one or more processors to further enable the user or the healthcare provider to book one of the types of vehicles in the fleet.

13. The non-transitory computer-readable medium of claim 9 for storing instructions that, when executed on one or more processors, cause the one or more processors to further:
access medical equipment types from a clinic database, wherein the clinic database stores medical equipment types, and wherein the transportation scheduling module is in communication with the clinic database, and based on the user credentials and current conditions, schedules the patient at one of a plurality of clinics.

14. The non-transitory computer-readable medium of claim 13, for storing instructions that, when executed on one or more processors, cause the one or more processors to further send a notification to the user and the clinic of a change of an appointment, a cancellation of the appointments, and via GPS, arrival of the user.

15. The non-transitory computer-readable medium of claim 9 for storing instructions that, when executed on one or more processors, cause the one or more processors to:
sorts data to transform the data for output at a virtual server;
training a training module to collect the data and update the machine learning module thereby providing a loop; and
optimize the system to analyze the data and reconfigure the transport server and clinic server to improve system performance.

16. A method for data and signal routing for scheduling and managing services incorporated in a system including a client device, and a server in communication with the client device, wherein the server comprises a memory to store instructions and a processor coupled with the memory to process the stored instructions, the method comprising the steps of:
generating, for display on a client device graphical user interface (GUI) associated with at least one user, an input screen that allows a user to input a request to a first service provider, wherein the first service provider is a healthcare clinic be completed by the user;
receiving the input from the client device GUI at an integrated server via a network, and in response, output a request to a second service provider, wherein the second service provider is a transport company;
receiving user personal credentials associated with the user;
outputting the patient credentials to a patient database via the network;
arranging, based on the input request and the personal credentials on the database, a clinic appointment for the user and a transportation type to the clinic for the user;
wherein the transportation type comprises one of a plurality of different types of vehicles in a vehicles fleet.

17. The method of claim 16, wherein the plurality of different types of vehicles in fleet comprise a car, a handicap accessible van, and an ambulance, wherein the vehicles are autonomous vehicles.

18. The method of claim 16, further comprising:
enabling the user to input a current conditions and a telehealth sub-module for communication with a physician or clinician, wherein the scheduling module utilizes current conditions to select the transportation type to the clinic for the user

19. The method of claim 16, further comprising enabling the user or the healthcare provider to book one of the types of vehicles in the fleet.

20. The method of claim 16, further comprising accessing medical equipment types from a clinic database, wherein the clinic database stores medical equipment types, and wherein the transportation scheduling module is in communication with the clinic database, and based on the user credentials and current conditions, schedules the patient at one of a plurality of clinics.
